SEQUENCE LISTING (8 sequences)

SEQ ID NO: 1 - DNA, 20 bp, Mus musculus
cttagccagtcccgaaacct

SEQ ID NO: 2 - DNA, 19 bp, Mus musculus
ttggtcccgtgtgatgtct

SEQ ID NO: 3 - DNA, 23 bp, Mus musculus
gttcaacatcaagagcagtagca

SEQ ID NO: 4 - DNA, 20 bp, Mus musculus
ctgcagacagagacgccatt

SEQ ID NO: 5 - DNA, 20 bp, Mus musculus
acagggcgttttatcttgcg

SEQ ID NO: 6 - DNA, 18 bp, Mus musculus
tccaagctcccggctaag

SEQ ID NO: 7 - DNA, 21 bp, Mus musculus
gctacagcttcaccaccacag

SEQ ID NO: 8 - DNA, 22 bp, Mus musculus
ggtctttacggatgtcaacgtc

(19) United States
(12) Patent Application Publication
(10) Pub. No.: US 2011/0256611 A1
(43) Pub. Date: Oct. 20, 2011

(54) INTERFERON BETA PRODUCTION PROMOTER AND A METHOD FOR PRODUCING THEREOF

(76) Inventors: Tadaomi Kawashima, Chiba (JP); Daisuke Kaneko, Chiba (JP); Ikuko Masuda, Chiba (JP); Noriko Tsuji, Ibaraki (JP); Akemi Kosaka, Ibaraki (JP)

(21) Appl. No.: 12/452,414

(22) PCT Filed: Jul. 3, 2008
(86) PCT No.: PCT/JP2008/062065

§ 371 Date: Jan. 26, 2010

(30) Foreign Application Priority Data
Apr. 3, 2007 (JP) 2007-174694
Nov. 19, 2007 (JP) 2007-299242

(51) Int. Cl. C12N 1/20; C07H 21/02

(52) U.S. Cl. 435/252.9; 435/252.1; 536/23.1; 435/253.4

(57) ABSTRACT

An interferon β production promoter and a method for producing thereof are provided. The interferon β production promoter has a culture, a cell or cell components of lactic acid bacteria as an active ingredient thereof. RNA can be used for the cell component. In addition, bacteria belonging to the genus Lactobacillus, Tetragenococcus, Pediococcus or Streptococcus can be used for the lactic acid bacteria. The interferon β production promoter can be produced by culturing lactic acid bacteria and collecting a culture, a cell or cell components thereof.

### TECHNICAL FIELD

The present invention relates to an interferon β production promoter and to a method for producing thereof.

### BACKGROUND ART

Interferon β is a physiologically active substance initially produced by immune cells in terms of activating antiviral function. Interferon β increases expressed amounts of interferon regulatory factor 7 in antigen-presenting cells such as dendritic cells and macrophages, resulting in the induction of production of interferon a and the production of various inflammatory cytokines such as interleukin 12, and the induction of Th1 immunity. This also results in induction of the production of interferon γ. On the basis thereof, interferon β is considered to be the most important substance in the induction of Th1 immunity.

When production of interferon β is promoted, induction of Th1 immunity, activation of immune cells and increased resistance to viruses occur. Thus, promoters of interferon β production can be used as immunoactivators, anti-type I allergics, therapeutic agents for hepatitis B and hepatitis C or cancer immunotherapeutic agents. Moreover, such promoters can also compensate for attenuation of immune functions accompanying aging.

Production of interferon α is known to be induced by ingestion of lactic acid bacteria (see, for example, Patent Document 1 and Non-Patent Document 1). In addition, production of interferon γ is known to be promoted by lactic acid bacteria belonging to the genus Tetragenococcus (see, for example, Patent Document 2). However, lactic acid bacteria are not known to promote production of interferon β.

Since interferon β has actions such as antitumor action, antiviral action or cell growth and differentiation regulatory action, it is used in the treatment of various diseases such as the treatment of cancer and the treatment of hepatitis B and C. Although interferon β is used by ingesting orally, oral ingestion makes it difficult for the interferon β to be absorbed into the body thereby preventing expectations of adequate effects. Moreover, continuous ingestion results in problems leading to adverse effects such as decreased leukocyte count, decreased platelet count, alopecia, fever, pain and fatigue. Consequently, there is a need to provide an interferon β production promoter that is highly safe and causes few adverse effects.
Patent Document 1: Japanese Unexamined Patent Publication No. H9-188627
Patent Document 2: Japanese Unexamined Patent Publication No. 2006-028047
Non-Patent Document 1: “Shokuhin to kaihatsu Vol. 42, No. 5”, 2007, p. 85-87

### DISCLOSURE OF THE INVENTION

### Problems to be Solved by the Invention

An object of the present invention is to provide an interferon β production promoter and a method for producing thereof.

### Means for Solving the Problems

The inventors of the present invention found that lactic acid bacteria belonging to various genii promote the production of interferon β, thereby leading to completion of the present invention. Namely, the invention relates to the following:

(1) an interferon β production promoter having for an active ingredient thereof a culture, a cell or cell components of lactic acid bacteria;
(2) the interferon β production promoter described in (1) above, wherein the cell component is RNA;
(3) the interferon β production promoter described in (1) or (2) above, wherein the lactic acid bacteria belong to the genus Lactobacillus, Tetragenococcus, Pediococcus, Streptococcus or Bifidobacterium;
(4) a food comprising the interferon β production promoter described in anyone of (1) to (3) above; and,
(5) a method for producing an interferon β production promoter comprising: culturing lactic acid bacteria and collecting a culture, a-cell or cell components thereof.

### EFFECTS OF THE INVENTION

An interferon β production promoter and a method for producing thereof are provided by the present invention. The interferon β production promoter can be preferably used as an immunoactivator, anti-type I allergic, hepatitis B or C therapeutic agent or cancer therapeutic agent.

### BRIEF DESCRIPTION OF THE DRAWINGS

FIG. 1 is a graph showing the results of a test of the promotion of interferon β production in peritoneal exudate macrophages by various lactic acid bacteria cells.

FIG. 2 is a graph showing the results of a test of the promotion of interferon β production in mouse bone marrow-derived dendritic cells by various lactic acid bacteria cells.

FIG. 3 is a graph showing the results of a test of the promotion of interferon β production in bone marrow-derived dendritic cells by lactic acid bacteria Tetragenococcus halophilus cells and RNaseA-treated cells.

FIG. 4 is a graph showing the results of a test of the promotion of interleukin 12 production in bone marrow-derived dendritic cells by lactic acid bacteria Tetragenococcus halophilus cells and RNaseA-treated cells.

FIG. 5 is a graph showing the results of a test of the promotion of interferon β production in TLR3-deficient bone marrow-derived dendritic cells by lactic acid bacteria Tetragenococcus halophilus cells.

FIG. 6 is a graph showing the results of a test of the promotion of interferon β production in TRIF-deficient bone marrow-derived dendritic cells by lactic acid bacteria Tetragenococcus halophilus cells.

FIG. 7 is a graph showing the results of a test of the promotion of interleukin 12 production in an interferon β test by lactic acid bacteria Tetragenococcus halophilus cells.

FIG. 8 is a graph showing the results of a test of the promotion of interleukin 12p35 mRNA expression by lactic acid bacteria Tetragenococcus halophilus cells.

FIG. 9 is a graph showing the results of a test of the promotion of interleukin 12p40 mRNA expression by lactic acid bacteria Tetragenococcus halophilus cells.

FIG. 10 is a graph showing the results of a test of the promotion of interferon regulatory factor 7 mRNA expression by lactic acid bacteria Tetragenococcus halophilus cells.

FIG. 11 is a figure showing the effects of increasing interferon γ-producing cells by lactic acid bacteria Tetragenococcus halophilus cells and inhibition thereof by anti-interferon β antibody.

### BEST MODE FOR CARRYING OUT THE INVENTION

### (1) Promotion of Interferon β Production

The interferon β production promoter of the present invention has for an active ingredient thereof a culture, a cell or cell components of lactic acid bacteria. Lactic acid bacteria refer to lactic acid bacteria belonging to, for example, the genus Lactobacillus, Tetragenococcus, Pediococcus or Streptococcus.

The active ingredient in the form of a culture, cells or cell component of lactic acid bacteria may be prepared by any method provided it demonstrates activity that promotes production of interferon β. Lactic acid bacteria cells are obtained by centrifugally separating a culture thereof followed by removal of the medium. A cell component refers to, for example, RNA produced by lactic acid bacteria, and more specifically, single-stranded RNA or double-stranded RNA. Lactic acid bacteria RNA can be obtained by ordinary fractionation methods.

In the case of ingesting the interferon β production promoter of the present invention for the purpose of enhancing immune activity or infection resistance, the amount ingested may be suitably set according to the symptoms and physique of the ingesting person. In the case of using cells of lactic acid bacteria belonging to the genus Tetragenococcus for the active ingredient, the ingested amount thereof is, for example, 1 to 1000 mg/60 kg of body weight/day.

The active ingredient in the form of a culture, a cell or cell components of lactic acid bacteria may also be used alone, or it can be used by adding to a food, drink or pharmaceutical.

### (2) A Method for Producing Interferon β Production Promoter

The interferon β production promoter of the present invention is obtained by culturing lactic acid bacteria and collecting a culture, a cell or cell components thereof. There are no particular limitations on the culturing conditions of the lactic acid bacteria, or the method used to collect the active ingredient in the form of a culture, a cell or cell components provided the active ingredient in the form of a component that demonstrates activity promoting production of interferon β is obtained.

The following indicates an example of a production process of an interferon β production promoter having for an active ingredient thereof cells of Pediococcus pentosaceus NRIC 1915 (acquired from the Noda Institute for Scientific Research Laboratory).

First, for example, Pediococcus pentosaceus is inoculated into MRS medium followed by culturing for 24 to 72 hours at 25 to 37° C. Cells are then collected by removing the medium after culturing with an ultrafiltration membrane or centrifugal concentrator. The resulting cells are washed with water or saline.

Cells, or a cell suspension consisting of cells suspended in water or saline, obtained in the manner described above can then be used as the interferon β production promoter of the present invention.

### Example 1

### Test of Promotion of Interferon β Production by Various Species of Lactic Acid Bacteria Using Mouse Peritoneal Exudate Macrophages

A test of the promotion of interferon β production was carried out using the lactic acid bacteria indicated in Table 1.

TABLE 1

| The genus of Lactic | |
|---|---|
| Acid Bacteria | Strains |
| Pediococcus | NRIC0099, NRIC0122, NRIC1914, |
| | NRIC1915 |
| Lactobacillus | NRIC0644, NRIC1067, NRIC1554, |
| | NRIC1559, NRIC1929, NRIC1930, |
| | NRIC1933 |
| Tetragenococcus | NBRC12172, NISL7116, NISL7118, |
| | NISL7119, NISL7126 |
| Leuconostoc | NRIC1539, NRIC1777, NISL7223 |
| Streptococcus | NRIC0256 |

Strains having the prefix NRIC were acquired from the Nodai Research Institute Culture Collection of the Tokyo University of Agriculture.
Strains having the prefix NBRC were acquired from the NITE Biological Resource Center of the National Institute of Technology and Evaluation.
Strains having the prefix NISL were acquired from the Noda Institute for Scientific Research Laboratory.

### 1. Preparation of Lactic Acid Bacteria Suspensions

Each of the lactic acid bacteria were inoculated into MRS medium at 1×10⁷ cells/ml. Tetragenococcus species were inoculated into MRS medium containing 10% salt at 1×10⁷ cells/ml. Following stationary culturing for 48 to 72 hours at 30° C., sterilization was carried out by boiling for 10 minutes at 95° C. Subsequently, the bacteria were collected by removing the media with a centrifugal concentrator. After washing the cells with physiological saline, the cells were suspended in RPMI medium for cell culturing so an optical absorbance at a wavelength of 600 nm of 0.125 to prepare lactic acid bacteria suspensions.

### 2. Test of Promotion of Interferon β Production

The interferon β production promoting activity of the prepared lactic acid bacteria suspensions was evaluated using peritoneal exudate macrophages collected and prepared from mice (8 to 12 weeks old, BALB/c, males, acquired from Charles River Laboratories).

### (1) Collection and Preparation of Peritoneal Exudate Macrophages

Peritoneal exudate macrophages were aseptically collected from mice three days following stimulation by administering 2 ml of thioglycolate into the abdominal cavity. After washing the collected macrophages with proprietarily prepared FBS solution, the number of cells was measured followed by preparation in RPMI-1640 medium to a concentration of 2×10⁶ cells/ml. The prepared macrophage cell solution was inoculated into a 96-well tissue culture plate at 100 μl per well. A hundred μl of RPMI-1640 medium or suspension of lactic acid bacteria adjusted to the concentration indicated above were added to each well followed by culturing in a 5% CO₂ incubator at 37° C., and the culture supernatant was collected at 3 hours and 6 hours after the start of co-culturing.

### (2) Measurement of Interferon β Production Promoting Activity

Culture supernatant collected over time as described above was measured by enzyme immunoassay using an interferon β assay kit (PBL).

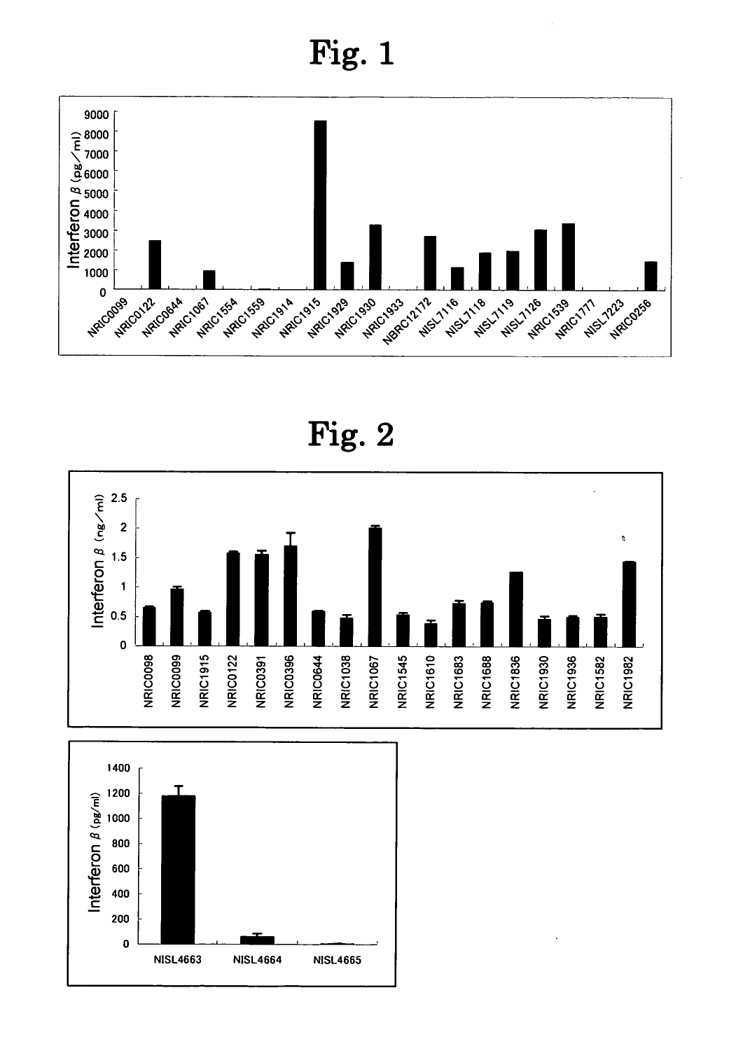

The results are shown in FIG. 1. Lactic acid bacteria belonging to the genii Tetragenococcus, Lactobacillus, Pediococcus, Leuconostoc and Streptococcus were confirmed to promote production of interferon β in peritoneal exudate macrophages. On the basis of the above, lactic acid bacterial cells were indicated to be useful as promoters of interferon β production.

### Example 2

### Test of Promotion of Interferon β Production in Various Lactic Acid Bacteria Using Bone Marrow-Derived Dendritic Cells

TABLE 2

| The genus of Lactic | |
|---|---|
| Acid Bacteria | Strains |
| Pediococcus | NRIC0099, NRIC0122, NRIC1915 |
| Lactobacillus | NRIC0391, NRIC0396, NRIC0644, |
| | NRIC1038, NRIC1067, NRIC1545, |
| | NRIC1610, NRIC1683, NRIC1688, |
| | NRIC1836, NRIC1930, NRIC1936 |
| Tetragenococcus | NRIC0098 |
| Leuconostoc | NRIC1582, NRIC1982 |
| Bifidobacterium | NISL4663, NISL4664, NISL4665 |

Strains having the prefix NRIC were acquired from the Nodai Research Institute Culture Collection of the Tokyo University of Agriculture.
Strains having the prefix NBRC were acquired from the NITE Biological Resource Center of the National Institute of Technology and Evaluation.
Strains having the prefix NISL were acquired from the Noda Institute for Scientific Research Laboratory.

### 1. Preparation of Lactic Acid Bacteria Suspensions

Each of the lactic acid bacteria were inoculated into MRS medium at 1×10⁷ cells/ml. Tetragenococcus species were inoculated into MRS medium containing 10% salt at 1×10⁷ cells/ml. Following stationary culturing for 48 to 72 hours at 30° C., sterilization was carried out by boiling for 10 minutes at 95° C. Subsequently, the bacteria were collected by removing the media with a centrifugal concentrator. After washing the cells with physiological saline, lactic acid bacterial suspensions were prepared by suspending in RPMI medium for cell culturing.

### 2. Test of Promotion of Interferon β Production

### (1) Preparation of Bone Marrow-Derived Dendritic Cell Suspensions

Bones were placed in a 6 cm dish containing RPMI-1640 medium (Sigma) supplemented with ice-cooled 1% fetal calf serum (FCS, inactivated) by sacrificing BALB/c or C57BL/6 mice by cervical dislocation under isoflurane inhalation anesthesia followed by removing the femur and tibia from the legs. The bone marrow cells were suspended after evacuating by injecting RPMI-1640 medium supplemented with 1% FCS. The resulting cell suspension was filtered with a cell strainer (40 μm, BD Falcon) followed by centrifuging for 5 minutes at 440×g.

After adding red blood cell lysis buffer (5 mL, 0.155 M NH₄Cl, 0.01 M Tris, pH 7.5) and allowing to stand for 5 minutes on ice, RPMI-1640 medium supplemented with 1% FCS (5 mL) was added followed by centrifuging and washing twice with RPMI-1640 medium supplemented with 1% FCS. An antibody cocktail (100 μL/10⁷ cells) consisting of phycoerythrin (PE)-labeled I-A antibody (Clone M5/144.14.2, BD Pharmingen, 0.2 mg/mL), PE-labeled anti-CD4 antibody (Clone GK1.5, BD Pharmingen, 0.2 mg/mL) and PE-labeled anti-CD8 antibody (Clone 53-6.7, BD Pharmingen, 0.2 mg/mL) each diluted 1000-fold with MACS running buffer, and rabbit IgG (50 μg/mL, Zymed), were added followed by allowing to stand undisturbed for 30 minutes on ice.

After washing once with the MACS running buffer, anti-PE magnetic beads (20 μL/10⁷ cells, Miltenyi) and MACS running buffer (80 μL/10⁷ cells) were added followed by allowing to stand undisturbed for 15 minutes at 4 to 8° C. After washing once with MACS running buffer equal to 20 times the amount of reaction solution, the cells were suspended in MACS running buffer (0.5 mL/10⁸ cells) followed by separating the negative fraction using an automatic magnetic separation system (Auto MACS, Miltenyi). The isolated cells were washed once with RPMI-1640 medium supplemented with 1% FCS followed by suspending in a base medium supplemented with granulocyte/macrophage colony stimulating factor (GM-CSF). The base medium consisted of the addition of 10% inactivated FCS (Hyclone) to RPMI-1640 medium supplemented with penicillin (100,000 U/L, Meiji Seika), streptomycin (100 mg/L, Meiji Seika), 2-mercaptoethanol (50 μM, Gibco), L-glutamic acid (2 mM, Nacalai-Tesque) and HEPES (20 mM, Dojin Chemical).

GM-CSF consisted of the addition of a culture supernatant of plasmocytoma X63-Ag8 inserted with mouse GM-CSF gene (J558L-GM-CSF) to the base medium at 10%. The cell solution was suspended in Trypan blue (Gibco), and after counting the number of cells using a hematocytometer, the cell solution was dispensed into a 6-well cell culturing plate (BD Falcon) to 1.2×10⁶ cells/4 mL/well) and cultured. 2 mL of medium were removed by aspiration on day 3 and day 6 after the start of culturing followed by the addition of 2 mL of base medium containing GM-CSF and harvesting the suspended cells on day 8 after the start of culturing in the form of immature dendritic cells (CD11c-positive cells: >85%). After washing the cells three times with RPMI-1640 medium supplemented with 1% FCS, the cells were suspended in base medium to obtain bone marrow-derived dendritic cell suspensions.

### (2) Measurement of Interferon β Production Promoting Activity

The two types of cell suspensions obtained in the manner described above were mixed with lactic acid bacteria suspensions at fixed ratios (number of bone marrow-derived dendritic cells: number of lactic acid bacteria=1:50) followed by co-culturing. The supernatant was recovered after culturing for 6 hours followed by measurement of interferon β concentration in the supernatant by enzyme immunoassay in the same manner as Example 1.

The results are shown in FIG. 2. As shown in the graph, production of interferon β was confirmed for each species of lactic acid bacteria as well as Bifidobacterium species.

### Example 3

[Test of Promotion of Interferon β Production by Tetragenococcus Species Lactic Acid Bacteria]

### 1. Preparation of Cell Suspension

Tetragenococcus halophilus Th221 was used for the lactic acid bacteria. This species is deposited at the International Patent Organism Depositary, National Institute of Advanced Industrial Science and Technology under the accession number FERM AP-21310.

First, Tetragenococcus halophilus Th221 was inoculated into MRS medium containing 10% salt at 1×10⁷ cells/ml. Following stationary culturing for 48 to 72 hours at 30° C., sterilization was carried out by boiling for 10 minutes at 95° C. After washing the cells with physiological saline, the cells were suspended in physiological saline at 1×10⁹ cells/ml to prepare a lactic acid bacteria suspension.

### 2. RNaseA Treatment

RNaseA (Sigma, Ribonuclease A from Bovine Pancreas) was added to the lactic acid bacteria suspension to a concentration of 10 μg/ml followed by incubating for 1 hour at 37° C. Subsequently, the cells were washed with physiological saline and again suspended in physiological saline at 1×10⁹ cells/ml to prepare a lactic acid bacteria suspension.

### 3. Test of Promotion of Interferon β Production

Interferon β production promoting activity and interleukin 12 production promoting activity of the lactic acid bacteria suspensions prepared in the manner described above were evaluated using bone marrow-derived dendritic cells collected and prepared from 6-week-old BALB/c mice (Japan SLC).

### (1) Preparation of Bone Marrow-Derived Dendritic Cell Suspensions

Bone marrow-derived dendritic cells were prepared in the same manner as Example 2 by sacrificing BALB/c mice by cervical dislocation under isoflurane inhalation anesthesia followed by removal of the femur and tibia from the legs.

### (2) Measurement of Interferon β Production Promoting Activity

The two types of cell suspensions obtained in the manner described above were mixed with the lactic acid bacteria suspension at a fixed ratio (number of bone marrow-derived dendritic cells:number of suspended cells 1:50) followed by co-culturing. The supernatant was collected over time and interferon β concentration in the supernatant was measured by enzyme immunoassay in the same manner as Example 1.

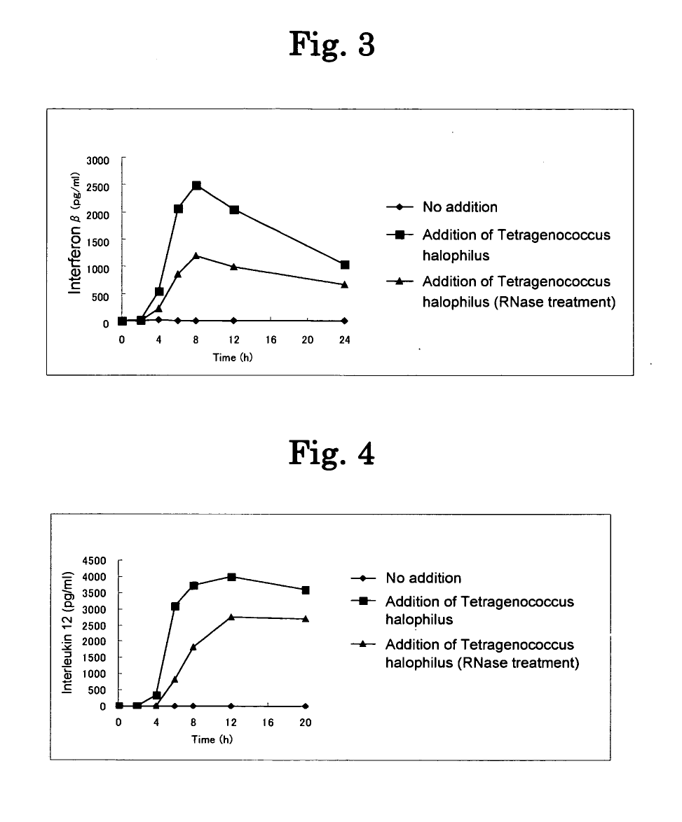

The results are shown in FIG. 3. The lactic acid bacteria suspension promoted interferon β production in bone marrow-derived dendritic cells, and interferon β production promoting activity decreased as a result of RNaseA treatment of the lactic acid bacteria suspension.

On the basis of the above, the active ingredient of interferon β production promoting activity was indicated to be RNA.

### 4. Measurement of Interleukin 12 Production Promoting Activity

The interleukin 12 concentration in the collected supernatant described above was measured by enzyme immunoassay. Enzyme immunoassay was carried out by adding 100 μl of a solution of rat anti-mouse interleukin 12 antibody (Pharmingen) adjusted to 2 μg/ml with 0.2 M, pH 6.0 phosphate buffer to each well of a 96-well tissue culture plate, and allowing to stand overnight at room temperature to allow the rat anti-mouse interleukin 12 antibody to adhere to the wells. Culture supernatant was then added at 100 μl/well followed by allowing to stand for 90 minutes at room temperature to allow the mouse interleukin 12 in the culture supernatant to bind to the rat anti-mouse interleukin 12 antibody adhered to the plate.

After washing the plate, rat biotinated anti-mouse interleukin 12 antibody (Pharmingen) was added to bind to the mouse interleukin 12 bound to the plate. After washing the plate, streptoavidin-labeled peroxidase enzyme (Vector) was added and bound to the biotin. TMB substrate solution (Moss/Cosmo Bio) was then added at 100 μl per well and allowed to react for 20 minutes at room temperature. After stopping the reaction with 0.5 N hydrochloric acid, absorbance at 450 nm was measured with a microplate reader, and the concentration of interleukin 12 in the culture supernatant was determined from a calibration curve prepared with recombinant mouse interleukin 12 (Pharmingen).

The results are shown in FIG. 4. A high degree of correlation was observed between interferon β and interleukin 12 production promoting activity, and production of interleukin 12, which is involved in induction of Th1 immunity together with interferon β, was induced. On the basis of the above, production of interferon β was confirmed to be promoted by stimulation of lactic acid bacteria, and immunoregulatory action was confirmed to be induced as a result thereof.

### Example 4

### Test of Promotion of Interferon β Production using TLR3-Deficient Mice

Interferon β production promoting activity was evaluated using mice in which TLR3 involved in recognizing double-stranded RNA was knocked out (6 to 12-week old C57BL/6 mice, females, acquired from the Hyogo College of Medicine) while focusing on Toll-like receptors (TLR) that recognize constituent components of lactic acid bacteria. Preparation of Bone Marrow-Derived Dendritic Cells, preparation of lactic acid bacteria, and measurement of interferon β production promoting activity were carried out using the same methods as Example 2. Interferon β concentrations were measured in the supernatant at 12 and 24 hours after the start of co-culturing.

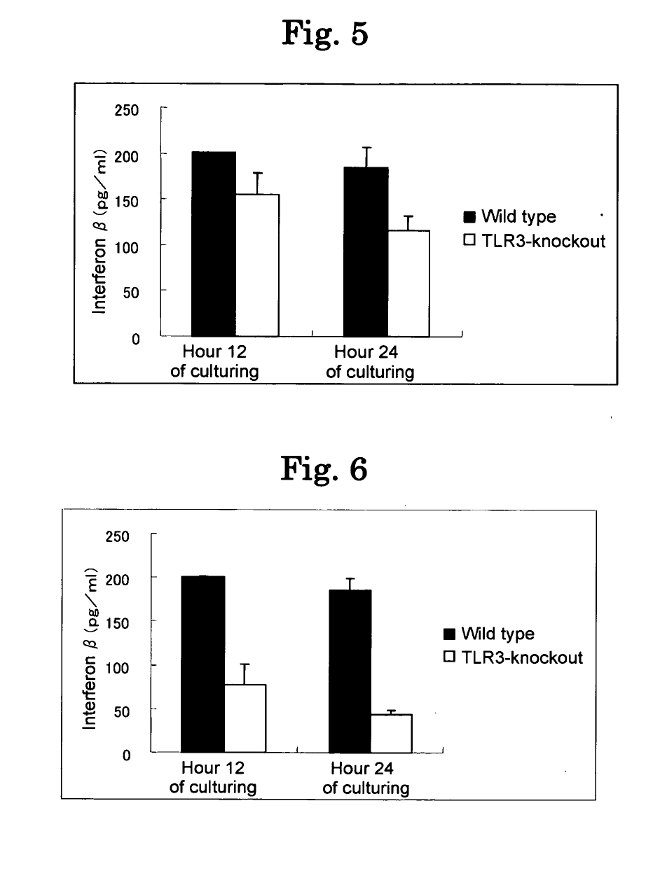

The results are shown in FIG. 5. Interferon β production promoting activity decreased in TLR3-deficient cells. On the basis of the above, TLR3 was indicated to be involved in interferon β production promoting activity, namely double-stranded RNA of lactic acid bacteria was shown to be involved in the active ingredient of interferon β production promoting activity.

### Example 5

### Test of Promotion of Interferon β Production Using TRIF-Deficient Mice

TRIF is an adapter molecule that transmits signals from TLR3. Interferon β production promoting activity was evaluated in the same manner as Example 3 using mice in which this adapter molecule had been knocked out (6 to 12-week old C57BL/6, females, acquired from the Hyogo College of Medicine). Preparation of bone marrow-derived dendritic cells, preparation of lactic acid bacteria, and measurement of interferon β production promoting activity were carried out using the same methods as Example 2. Interferon β concentrations were measured in the supernatant at 12 and 24 hours after the start of co-culturing.

The results are shown in FIG. 6. Interferon β production promoting activity decreased in TRIF-deficient cells. On the basis of the above, the interferon β production promoting activity of lactic acid bacteria was confirmed to be mediated by TRIF.

### Example 6

### Food Production Example

After culturing Pediococcus pentosaceus strain NRIC1915 in MRS medium for 24 hours at 30° C., the cells were dispersed in physiological saline and centrifuged to obtain a bacterial cell paste.

This paste was then added to unadulterated soy milk (Kibun Food Chemifa) and cultured for 24 hours at 30° C. to obtain a yogurt-like food.

### Example 7

### Interferon β Neutralization Test

Since a high correlation was confirmed to exist between interferon β and interleukin 12, rat-derived anti-mouse interferon β antibody (Yamasa) was added to a culture broth to a concentration of 80 μg/ml followed by measurement of the amount of interleukin 12 produced when interferon β secreted into the culture supernatant was neutralized by bone marrow-derived dendritic cells.

Preparation of Bone Marrow-Derived Dendritic Cells, preparation of lactic acid bacteria and measurement of interleukin β production promoting activity were carried out in the same manner as Example 3.

Expressed amounts of interleukin 12p35, interleukin 12p40 and interferon regulatory factor 7 mRNA were measured by quantitative real-time PCR.

Extraction of mRNA was carried out using the RNeasy Mini Kit (Qiagen) at 6 hours after the start of co-culturing. Subsequently, after dispensing the extract so that the amount of total RNA was 300 ng, a reverse transcription reaction was carried out using the ExScript RNA PCR Kit (Takara) to obtain cDNA.

Quantitative real-time PCR was then carried out with Sybr Premix Ex Taq (Takara) using this cDNA. Primers specific for each mRNA consisted of the sense strand 5′-CTTAGCCAGTCCCGAAACCT-3′ (SEQ ID NO. 1) and the antisense strand 5′-TTGGTCCCGTGTGATCTCT-3′ (SEQ ID NO. 2) for interleukin 12p35, the sense strand 5′-GTTCAACATCAAGAGCAGTAGCA-3′ (SEQ ID NO. 3) and the antisense strand 5′-CTGCAGACAGAGACGCCATT-3′ (SEQ ID NO. 4) for interleukin 12p40, the sense strand 5′-ACAGGGCGTTTTATCTTGCG-3′ (SEQ ID NO. 5) and the antisense strand 5′-TCCAAGCTCCCGGCTAAG-3′ (SEQ ID NO. 6) for interferon regulatory factor 7, and the sense strand 5′-GCTACAGCTTCACCACCACAG-3′ (SEQ ID NO. 7) and the antisense strand 5′-GGTCTTTACGGATGTCAACGTC-3′ (SEQ ID NO. 8) for β-actin, and measurements were carried out with the Mx3000P (Stratagene). Each expressed amount was standardized to the expressed amount of β-actin, and the relative expressed amount was determined based on the amount expressed prior to stimulation.

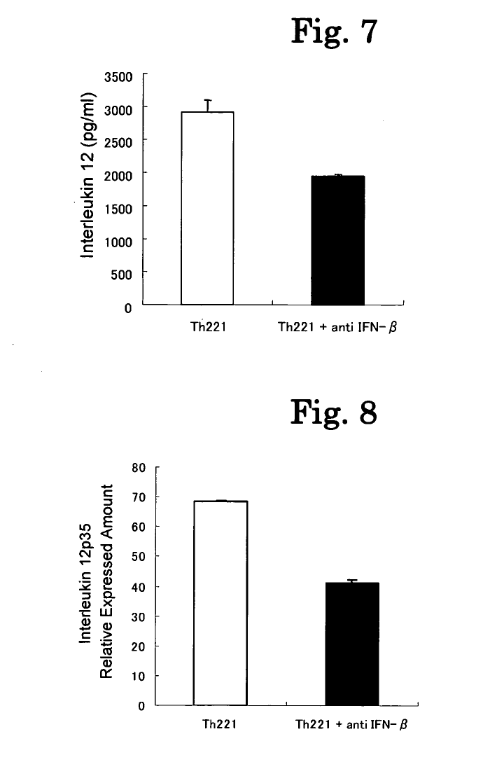

The amount of interleukin 12 produced decreased by neutralizing interferon β. The results are shown in FIG. 7.

Since the expressed amount of mRNA decreased for interleukin 12p35, interferon β was demonstrated to affect interleukin 12p35. The results are shown in FIG. 8.

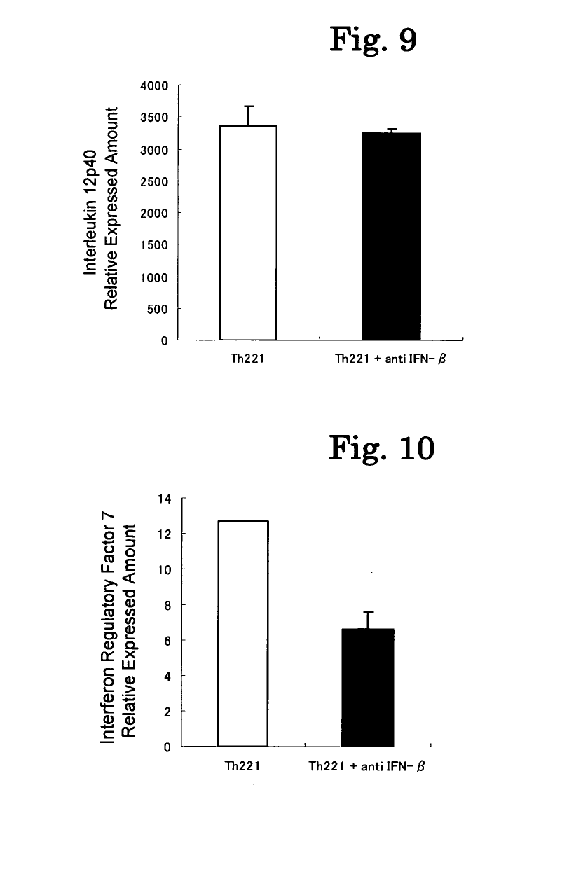

On the other hand, there was no effect on the expressed amount of interleukin 12p40. This result is shown in FIG. 9.

In addition, the expressed amount of interferon regulatory factor 7 induced by interferon β also decreased. That result is shown in FIG. 10.

On the basis of the above, interferon β produced by bone marrow-derived dendritic cells due to stimulation by Tetragenococcus halophilus Th221 was indicated to be involved in promoting the production of interleukin 12 as a result of inducing production of interleukin 12p35.

### Example 8

[Interferon β-Mediated Induction of Interferon γ-Producing Cells by Lactic Acid Bacteria]

A test was conducted to determine whether or not interferon γ-producing cells are induced by interferon β production promoting action of lactic acid bacteria during co-culturing of bone marrow-derived dendritic cells and CD4⁺ T cells.

Preparation of Bone Marrow-Derived Dendritic Cells and preparation of lactic acid bacteria were carried out in the same manner as Example 3. In addition, CD4⁺ T cells were prepared from the spleens of D011.10 mice. Spleens were collected from D011.10 mice and then shredded with a mesh to obtain spleen cells. Subsequently, the spleen cells were incubated for 30 minutes with anti-mouse CD4 beads (Miltenyi) to obtain CD4⁺ T cells using the Auto MACS system (Miltenyi).

1×10⁵ bone marrow-derived dendritic cells, 5×10⁵ CD4⁺ T cells and 5×10⁶ Tetragenococcus halophilus Th221 per well were cultured in a 96-well plate. Rat-derived anti-mouse interferon β antibody (Yamasa) was added to the culture broth at 80 μg/ml to neutralize the interferon β secreted into the culture supernatant by the bone marrow-derived dendritic cells. Anti-rat IgG1 antibody was used for the control antibody.

The medium was replaced on day 3 from the start of culturing, and the proportions of interferon γ-producing cells and interleukin 4-producing cells were investigated using the FACS Aria flow cytometry system (BD) on day 7. Anti-mouse interferon γ antibody (BD Pharmingen) and anti-mouse interleukin 4 antibody (BD Pharmingen) were used for measurement.

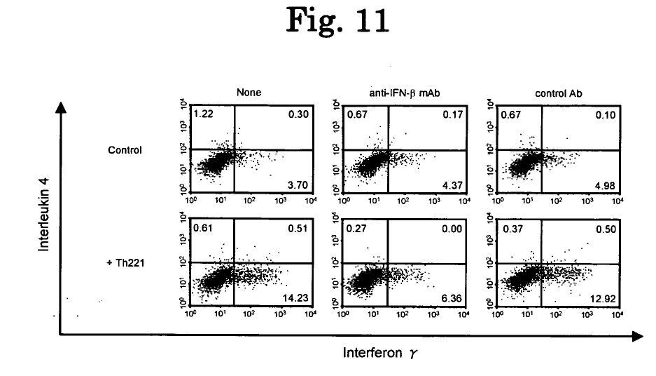

The results are shown in FIG. 11. The proportion of interferon γ-producing cells increased due to stimulation by Tetragenococcus halophilus Th221. In addition, induction of interferon γ-producing cells was inhibited by anti-interferon β antibody. On the basis thereof, interferon γ-producing cells were confirmed to be induced through mediation by interferon p due to stimulation by lactic acid bacteria.

### INDUSTRIAL APPLICABILITY

An interferon β production promoter is provided by the present invention. This interferon β production promoter can be used as an immunoactivator, anti-type I allergic, hepatitis B or C therapeutic agent or cancer therapeutic agent.

1. An interferon β production promoter having for an active ingredient thereof a culture, a cell or cell components of lactic acid bacteria.

2. The interferon β production promoter according to claim 1, wherein the cell component is RNA.

3. The interferon β production promoter according to claim 1, wherein the lactic acid bacteria belong to the genus Lactobacillus, Tetragenococcus, Pediococcus or Streptococcus.

4. A food comprising the interferon β production promoter according to claim 1.

5. A method for producing an interferon β production promoter comprising: culturing lactic acid bacteria and collecting a culture, a cell or cell components thereof.

6. The interferon β production promoter according to claim 2, wherein the lactic acid bacteria belong to the genus Lactobacillus, Tetragenococcus, Pediococcus or Streptococcus.

7. A food comprising the interferon β production promoter according to claim 2.

8. A food comprising the interferon β production promoter according to claim 3.

9. A food comprising the interferon β production promoter according to claim 6.
